SEQUENCE LISTING (3 sequences)

SEQ ID NO: 1 - protein, 16 aa, Homo sapiens
LNFARKRIKNPEGGLC

SEQ ID NO: 2 - protein, 60 aa, Homo sapiens
QTAESLAVHNITVCEQKISEWGKIKECPIPQFAGNPPMTWKWIVGPMFLYLCERLVRFWR

SEQ ID NO: 3 - protein, 45 aa, Homo sapiens
AERIVRGQTAESLAVHNITVCEQKISEWGKIKECPIPQFAGNPPM

(19) United States
(12) Patent Application Publication
(10) Pub. No.: US 2012/0156704 A1
(43) Pub. Date: Jun. 21, 2012

(54) IN VITRO METHOD FOR DETECTING GP91PHOX AS A MARKER OF OXIDATIVE STRESS

(75) Inventors: Francesco Violi, Rome (IT); Pasquale Pignatelli, Rome (IT)

(73) Assignee: BIOS INTERNATIONAL S.R.L., Rome, RM (IT)

(21) Appl. No.: 13/377,559

(22) PCT Filed: Jun. 11, 2010
(86) PCT No.: PCT/EP10/58253

§ 371 Date: Mar. 6, 2012

(30) Foreign Application Priority Data
Jun. 12, 2009 (IT) RM2009AOOO302

(51) Int. Cl. G01N 33/53; C12N 5/16; C07H 21/04; C07K 16/18; C07K 7/08; C07K 14/46

(52) U.S. Cl. 435/7.92; 530/326; 530/324; 536/23.5; 530/387.9; 435/326; 530/388.2; 435/7.1

(57) ABSTRACT

The invention relates to an in vitro method for detecting the activation of NADPH oxidase by measuring gp91^{phox} protein levels in biological fluids, as a marker of oxidative stress. The method is useful for testing the oxidative stress levels in dysmetabolic pathologies, such as diabetes, hypercholesterolemia and hyperlipidemia, in pathologies of the cardiovascular district, such as hypertension, atherosclerosis, cardiac hypertrophy and stroke, and in clinical conditions comprising sepsis and diseases with a strong inflammatory component.

### FIELD OF THE INVENTION

The present invention relates to a non-enzymatic in vitro method for detecting gp91^{phox} protein in biological fluids as a marker of NADPH oxidase activation and of oxidative stress.

### BACKGROUND OF THE INVENTION

The reactive oxigen species (ROS) are small highly reactive molecules which are present in the human and animal body and which originate from oxygen. The ROS can modify cell functions by reacting with DNA, protein and lipid molecules, and are normally present in biologic system as by-products of different metabolic pathways. They also have a key role in cell signal transduction and in the regulation of immune system cellular activities.

Oxidative stress is defined as a serious imbalance in the production and presence of ROS with respect to the available cell antioxidant potential. It can lead to a serious physiological damage and, thus, it can contribute to the onset of a pathology. Potentially, any cell component can be involved and damaged by oxidative stress, such as DNA, carbohydrates and proteins. A number of metabolic diseases are characterized by the presence of a marked oxidative stress, among which diabetes, hypercholesterolemia, hyperlipidemia, and cardiovascular conditions, such as hypertension, atherosclerosis, cardiac hypertrophy and myocardial infarction. High levels of oxidative stress are also detected in sepsis and in diseases with a strong inflammatory component, such as rheumatoid arthritis (Cave A. C. et al., Antiox. Redox Signal. (2006) 8:691-728; Valko M. et al., Int. J. Biochem. Cell. Biol. (2007) 39:44-84).

Oxidative stress per se is a difficult phenomenon to be tackled and measured in vivo. This is because ROS are very short lived, and cannot be detected in blood or tissues. Therefore, their levels could only be measured by indirect methods, such as detection of their derivatives in cellular DNA or in aminoacids, but a precise correlation between these by-products of cellular ROS production and actual oxidative stress has not been proven yet (Halliwell B. and Whiteman M., Br. J. Pharmacol. (2004) 142:231-255). A method frequently used in vitro is the detection of ROS by use of fluorescent probes such as acetate of dichlorofluorescein acetate (DCFH), dihydrorhodamine (DHR), luminol and lucigenin, but it cannot be translated into a method for in vivo detection.

Lipid peroxidation is a rather complex process which generates a number of different compounds in very variable quantities. Notwithstanding this, detection of such generated compounds is still considered as a useful index of oxidative stress.

Oxidative stress levels in vivo are currently measured by EIA or ELISA detection of isoprostanes in urines (Wang Z. et al., Pharmacol. Exp. Ther. (1995) 275:94-100; U.S. Pat. No. 6,620,800; U.S. Pat. No. 5,700,654, U.S. Pat. No. 5,891,622), or either by mass spectrometry (M. S. Lawson et al., J. Biol. Chem. (1999) 274:2441-2444). Isoprostanes are produced in our body by means of direct peroxidation of arachidonic acid (AA) with no intervention of any enzymatic process, and are thus considered as a direct measure of peroxidation in vivo.

A pivotal role in the production of ROS by cells is played by the enzyme NADPH oxidase, a multi-subunit protein originally discovered in phagocytic cells. NADPH oxidase comprises 5 subunits, of which the catalytic one is a membrane protein known as Nox (of which 5 different subtypes are known). In immune phagocytic cells, such as neutrophils and macrophages, the subtype of Nox present is Nox2, also known as gp91^{phox}, where it is responsible for the production of superoxide anion and, secondarily, of oxygen peroxide, which allow these cells to kill bacterial cells during infections. Phagocytic NADPH oxidase gets activated when gp91^{phox} interacts primarily with a second membrane subunit of the enzyme, i.e. p22^{phox}, and subsequently the remaining subunits normally present in the cytoplasm, p47^{phox}, p67^{phox} e Rac1-2 are recruited on the plasma membrane to reconstitute the working enzyme (Sheppard F R et al., J Leukoc Biol (2005) 78:1025-1042).

A role for ROS in the regulation of vasodilation, in atherosclerosis and in the process of inflammation has been established in the literature (Dworakowsky R. et al., Pharmacol. Reports (2008) 60: 21-28; Martino F. et al., Pediatrics (2008) 122:e648-e655; Bedard K and Krause K H, Physiol rev (2007) 87: 245:313; Valko M et al., Int J Biochem Cell Biol (2007) 39:44-84; Bauerova K and Bezek S. (1999) Gen Physiol. Biophys. 18 (spec. issue):15-2); Griffiths H. R. (2008) Autoimmun. Rev 7: 544-449; Carnevale R. et al (2007) FASEB J. 21: 927-934; Newsholme P. et al. (2007) J. Physiol. 583: 9-24) By way of example, in hypercholesterolemia the excess presence of LDL (low density lipoprotein) in blood causes an increase in NADPH oxidase in platelets and vascular wall cells and significantly contributes to the infiltration of foam cells and to the formation of atheromatic plaques.

In the literature, the activation of NADPH oxidase is never measured. Actually, the presence of gp91^{phox} has been detected in cells (cultured or not) and tissues (Vaziri et al., Biochim. Biophys. Acta (2005) 1723: 321-327; Wolfort et al., Am. J. Physiol. Heart Circ Physiol. (2008) 294: H2619-H2626; Paravicini T. M. et al., Circ. Res. (2002) 91:54-61; Morawietz et al., Biochem. Biophys. Res Commun. (2001) 285:1130-1135; Samuelson D. J. et al., J. Leukoc. Biol. (2001) 69:161-168; Anrather J. et al., J. Biol. Chem. (2006) 281:5657-5667; Gandhi M. S. et al., J. Cardiovasc Pharmacol. (2008) 52:245-252).

In the cases mentioned above the quantitative measure of the presence of its subunits, in particular of the catalytic subunit gp91^{phox}, by means of PCR methods, flow cytometry or immunoblotting, or an indirect testing of the enzymatic activity of this enzyme by measuring the levels of ROS produced in certain conditions in supernatant or cell cultures, were considered to be indicative of NADPH activity (Cross A. R., Ericson R. W., and Curnutte J. T., Biochem. J. (1999) 341:251-255; Teufelhofer O. et al., Toxicol. Sci. (2003) 76:376-383).

Anyway, said methods, beside giving no information about NADPH oxidase activation but only about its presence in the samples, would not be particularly useful in the clinical setting, being complex, delicate and requiring dedicated and specialized staff to be carried out. In addition, they are time-consuming and not very useful for the analysis of a large number of samples.

The analysis of the presence of gp91^{phox} in platelets, for example, requires that these blood elements are extracted in a very short time from the sample, i.e. within 3 hours. Moreover, handling of platelets is very tricky, as it is mandatory that they are not activated or damaged in order to obtain reliable data.

WO 2007/047796 only furnishes a general disclosure which does not include any data or hint whatsoever to the fact that the presence of gp91^{phox} in serum is an index of NADPH oxidase activation.

As of now, thus, it is still impossible to connect ROS production (and oxidative stress) in a patient to the actual activation of his/her NADPH oxidase in vivo. In addition, not much is known about the steps following activation of platelet NADPH oxidase in the cell and the state of the enzyme itself after activation has been elicited.

Carnevale R. et al., supra, found that platelets isolated from hypercholesterolemic subjects can produce ROS and are able to efficiently oxidate LDL by means of NADPH oxidase ex vivo, while platelets from patients affected by ereditary lack of gp91^{phox} were not able to do so.

Finally, Martino F. et al., Pediatrics (2008) 122:e648-e655 found a weak correlation between the presence of gp91^{phox} on platelets from hypecholesterolemic pediatric patients and urinary isoprostane levels. As it will be clear to the expert in the field, the simple detection of a variation in the levels of an enzyme is not a measure of its activity, since the latter can be influenced by many factors that go beyond the simple increase in enzyme (or enzyme subunit) levels.

It may, therefore, be safely said that until now no one has been able to find an easy and direct way to measure the actual activation of NADPH oxidase in vivo or in a cultured cell system.

In addition, until now nobody has been able to establish a strong and significant relationship in vivo between actual activation of a pivotal enzyme for the production of ROS such as NADPH oxidase, and parameters indicative of oxidative stress, as, for example, the levels of urinary isoprostanes. Therefore, it is not possible as of now to monitor the activation of NADPH oxidase in vivo or in cells and to consider it as a straightforward, easy and reliable method for determining the oxidative stress levels in a patient or in a cultured cell system.

Also, it would be very useful if such a detection of NADPH oxidase activation could be carried out by means of a simple, unexpensive analytical method, e.g. an ELISA method, using starting samples such as plasma or serum, instead of cells or biopsies.

### SUMMARY OF THE INVENTION

The Inventors have now unexpectedly found that gp91^{phox} protein is present in human serum, and that said presence is a dependable index of NADPH oxidase activity, as the activation level of cell NADPH oxidase significantly correlates with the presence of gp91^{phox} in serum. They also found that NADPH activation levels significantly influence the concentration of urinary isoprostanes. Thus, assay of the gp91^{phox} levels in serum (indicated as sgp91^{phox}) can be also considered as a significant measure of body oxidative stress.

Therefore, it is an object of the present invention a simple, efficient and reliable analytical method which allows in vitro detection of gp91^{phox} protein in serum as a marker of NADPH oxidase activation in platelets and, at the same time, of oxidative stress, as shown in the following Example section. In particular, the method of the invention allows detection of soluble gp91^{phox} in serum and in other biological samples, such as plasma and cell culture supernatants, as a significant measure of the enzyme NADPH oxidase activation.

Still a further object of the invention is the evaluation of the efficacy of a given therapy, e.g., in case of sepsis, in diabetic patients or in hypercholesterolemic patients, by means of detection of the levels of gp91^{phox} in biological samples such as serum or plasma as a marker of NADPH oxidase activation and oxidative stress during said therapy.

A further object of the present invention is use of gp91^{phox} or corresponding peptide fragments for detecting activation of cellular NADPH oxidase and of the linked oxidative stress, in biological samples, such as, for example, serum, plasma or cell culture supernatants.

A further object of the invention are the peptides corresponding to SEQ ID NO 1, and to SEQ ID NO 3 and the corresponding peptide fragments and encompassing peptide sequences, which are used as a marker of NADPH oxidase activation.

A further object of the present invention are the polynucleotide sequences encoding for the peptides of SEQ ID NO 1 and of SEQ ID NO 3 and transcription and expression vectors, cDNAs, and RNAs comprising said polynucleotide sequences encoding for the peptides of SEQ ID NO 1 and of SEQ ID NO 3.

A further object of the invention are the polyclonal and the monoclonal antibodies against gp91^{phox} protein and its corresponding peptide fragments of SEQ ID NO 1 and of SEQ ID NO 3 for the detection of the presence of said protein in biological fluids as a marker of NADPH oxidase activation and oxidative stress.

A further object of the invention is a kit for the detection and measurement of gp91^{phox} protein, in particular its extracellular moiety that is released in serum and in different biological samples, as a marker of NADPH oxidase activation. The kit of the invention is particularly useful in the field of oxidative stress control for clinical purposes, such as in cardiovascular disease, dysmetabolic disease, inflammation and sepsis.

An additional object is the monoclonal antibody against the peptide of SEQ ID NO. 3 from hybridoma SD-6311 deposited at American Type Culture Collection (ATCC) on Jun. 10, 2010.

Further objects will be evident from the detailed description and the appended set of claims.

### BRIEF DESCRIPTION OF THE FIGURES

FIG. 1: Detection of gp91^{phox} in serum.

Panel A: To prove that gp91^{phox} can be detected as a soluble protein in blood, sera from 3 healthy patients were immunoprecipitated. Samples 1, 3 and 5 were immunoprecipitated with the monoclonal antibody against the peptide QTAESLAVHNITVCEQKISEWGKIKECPIPQFAGNPPMTWKWIVGP MFLYLCERLVRFWR (SEQ ID NO 2) of human gp91^{phox} (Santa Cruz Biotechnology, Inc.). Samples 2, 4 and 6 were precipitated with a non-specific antibody against goat IgG. The molecular weight of the bands is reported.

Panel B: Quantitative analysis of the immunoprecipitates of Panel A. The quantitative analyses of gp91^{phox} were performed by densitometry using the program “NIH IMAGE 1.63”.

FIG. 2: gp91^{phox} levels are increased in hypercholesterolemic patients.

Panel A: Quantitative analysis of gp91^{phox} serum levels in 30 hypercholesterolemic patients (HC) and 20 healthy subjects (HS) enrolled in the clinical study evaluated by western blot analysis. The quantitative analyses of the gp91^{phox} were performed by densitometry using the program “NIH IMAGE 1.63”.

Panel B: A representative. Western blot analysis of 10 hypercholesterolemic patients and of 10 healthy patients from the two groups of Panel A. The monoclonal antibody against the peptide QTAESLAVHNITVCEQKISEWGKIKECPIPQFAGNPPMTWKVVIVGP MFLYLCERLVRFWR (SEQ ID NO 2) of human gp91^{phox} (Santa Cruz Biotechnology, Inc.) was used for this analysis.

Panel C: Statistical analysis of the distribution of gp91^{phox} levels on platelets detected by citofluorimetry using the same monoclonal antibody as used in Panels A and B

Panel D: Statistical analysis of urinary isoprostane levels detected in the same 30 hypercholesterolemic patients and 20 healthy subjects of Panels A, B, and C.

FIG. 3: Clinical study in hypercholesterolemic patients.

Panel A: sgp91^{phox} serum levels measured by ELISA using the rabbit polyclonal antibody of the invention in HC patients randomized to be treated with a 30-day therapy with atorvastatin (10 mg/day) together with a proper diet, or to treatment with proper diet only.

Panel B: sgp91^{phox} serum levels in the same samples of Panel A measured by ELISA using the monoclonal antibody of the invention (from hybridoma SD-6311) at baseline and after 30 days of treatment.

Panel C: Presence of gp91^{phox} on the surface of platelets from the two treatment groups at baseline and after 30 days of treatment, as measured by flow cytometry.

Panel D: Urinary isoprostanes levels in the two treatment groups at baseline and after 30 days of treatment.

FIG. 4: ELISA standard curves obtained by using the primary antibodies and the corresponding immunizing peptides of the invention.

Panel A: ELISA standard curve obtained by using 64 pg/ml; 32 pg/ml; 16 pg/ml; 8 pg/ml of the immunizing peptide LNFARKRIKNPEGGLC (SEQ ID NO 1) from gp91^{phox} protein sequence, and the rabbit policlonal antibody of the invention.

Panel B: ELISA standard curve obtained by using 64 pg/ml; 32 pg/ml; 16 pg/ml; 8 pg/ml of the immunizing peptide AERIVRGQTAESLAVHNITVCEQKISEWGKIKECPIPQFAGNPPM (SEQ ID NO 3) from gp91^{phox} protein sequence, and the monoclonal antibody of the invention from hybridoma SD-6311

FIG. 5: Specific platelet NADPH oxidase activation is linked with its presence in soluble form in supernatants and serum.

Panel A: Platelet NADPH oxidase activation tested by measuring ROS production upon stimulation with arachidonic acid (AA) and in the presence of the specific inhibitors apocynin and gp91ds-tat and of the drug atorvastatin. (n=5; *p<0.001).

Panel B: western blot of platelet membranes gp91^{phox} from platelets activated with AA and in the presence of the specific inhibitors apocynin and gp91ds-tat and of the drug atorvastatin. The monoclonal antibody of the invention was used.

Panel C: Levels of gp91^{phox} in the supernatants of platelets activated with AA and in the presence of the specific inhibitors apocynin and gp91ds-tat and of the drug atorvastatin measured by ELISA assay using the monoclonal antibody of the invention. (n=5; *p<0.001).

Panel D: sgp91phox in serum, in AA-stimulated and unstimulated platelets, in phorbol-myristate acetate (PMA)-stimulated and unstimulated PMN and lipolysaccharide (LPS)-stimulated and unstimulated lymphocytes/monocytes (n=5; *p<0.001). The samples are the same as described above.

### DETAILED DESCRIPTION OF THE INVENTION

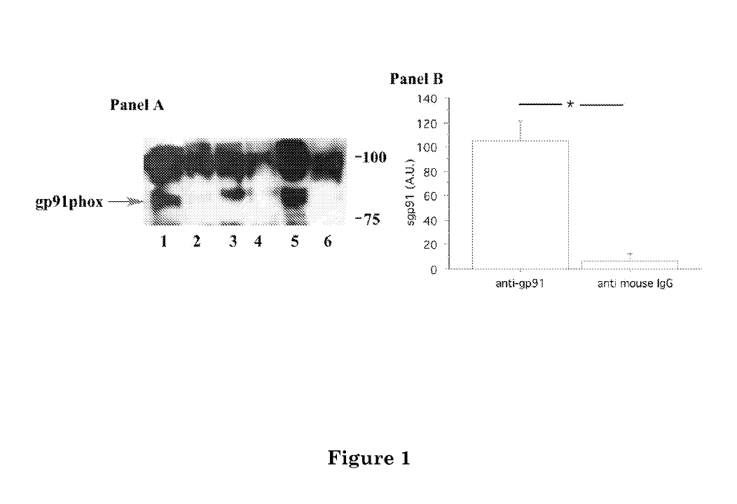

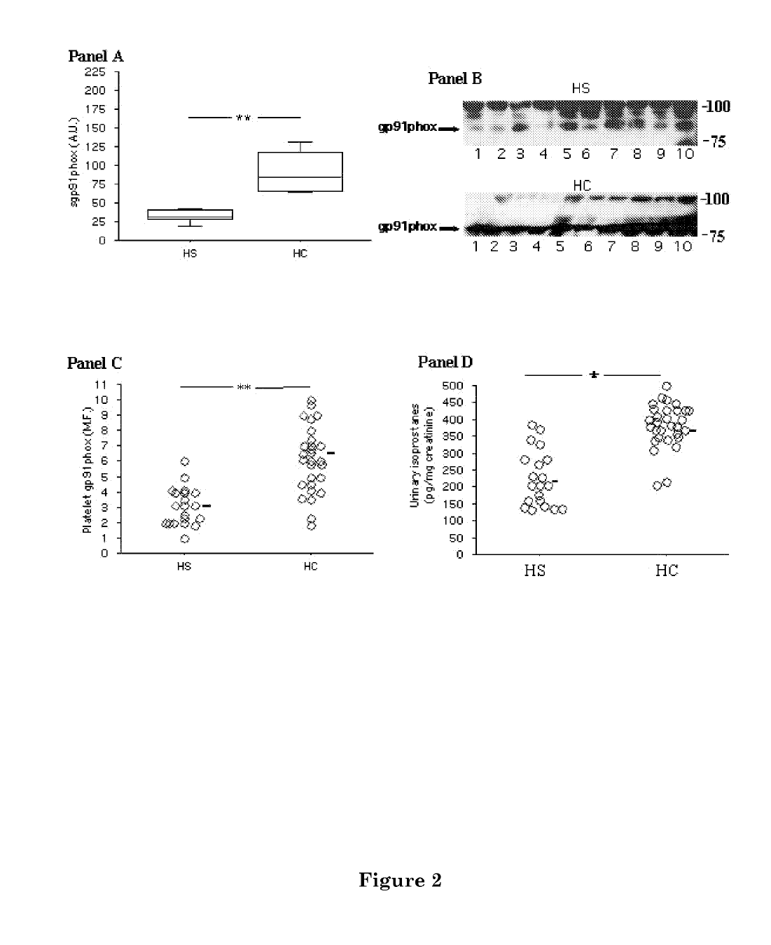

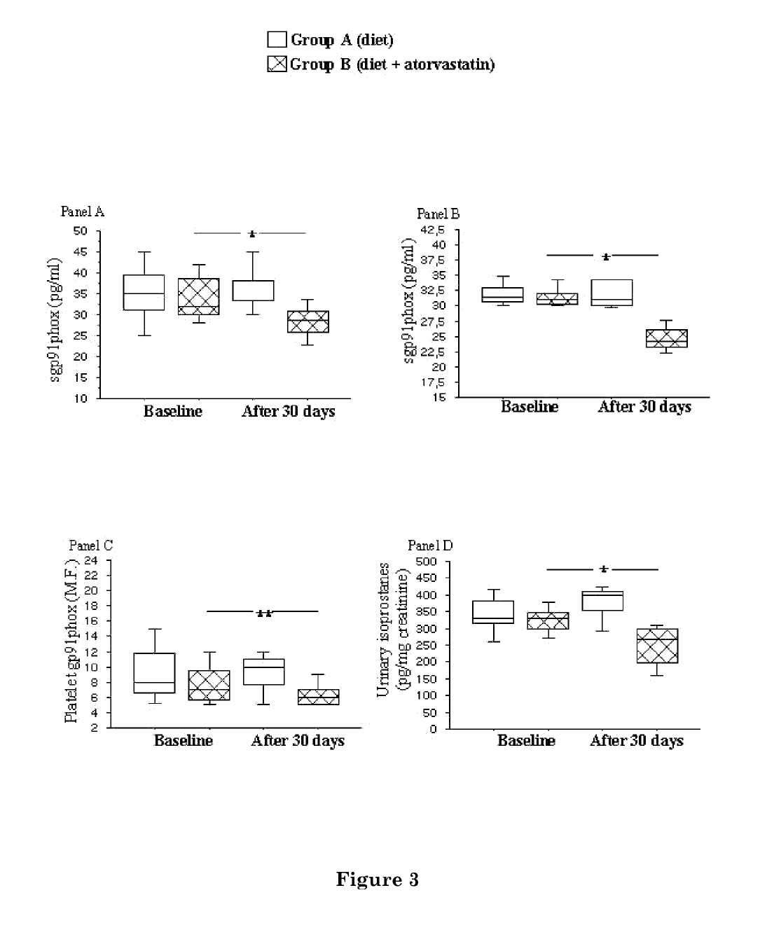

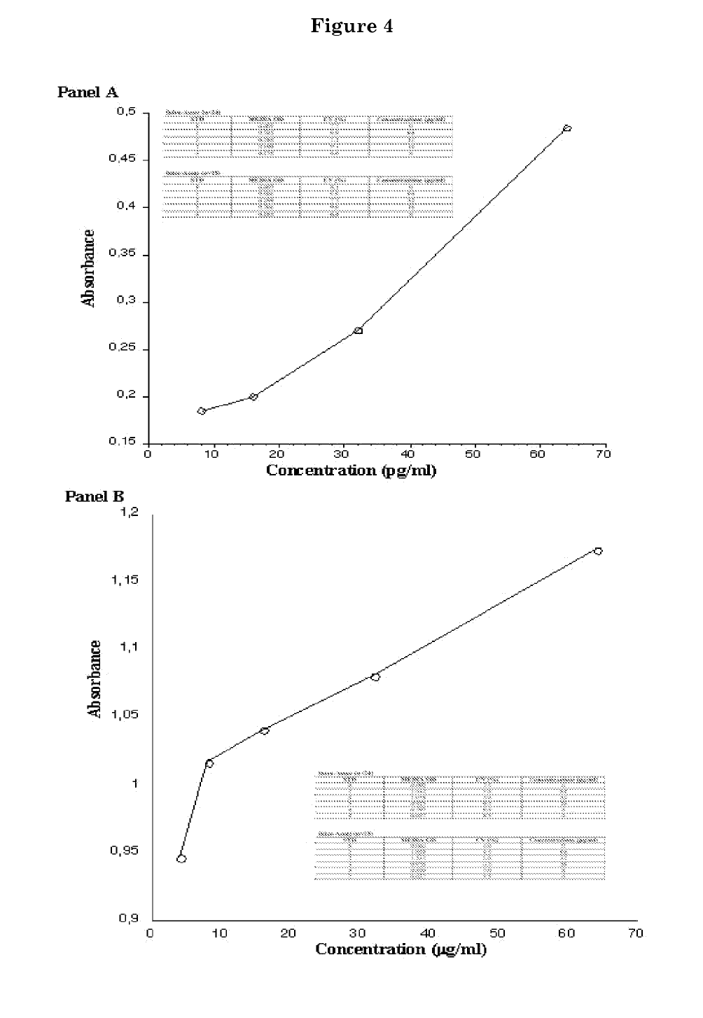

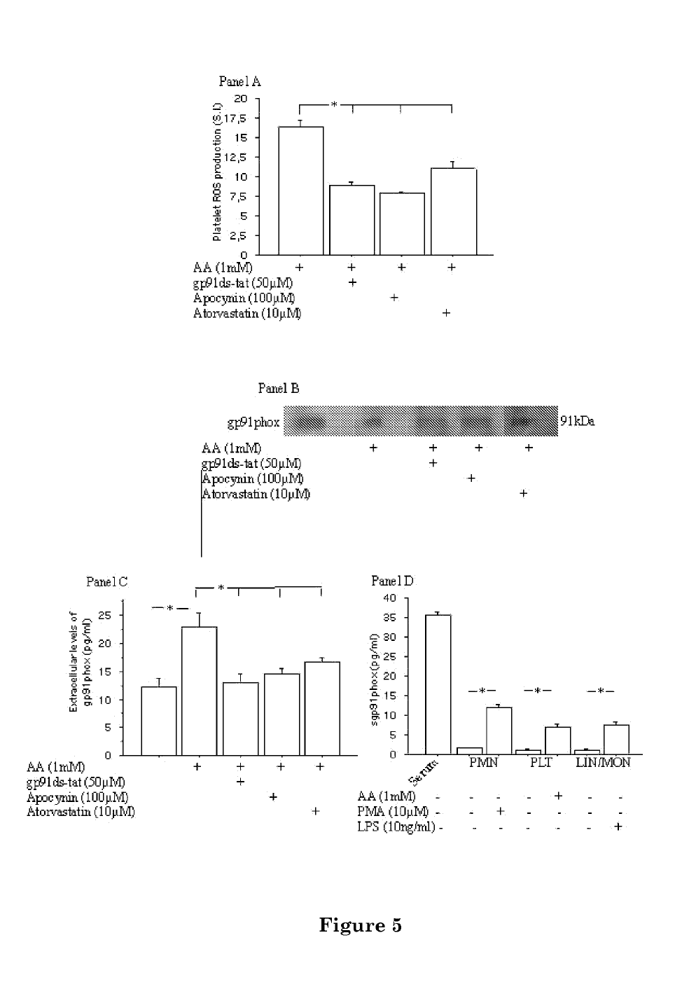

The present invention is based on the unexpected finding that, upon activation of the platelet enzyme NADPH oxidase, its catalytic subunit, the protein gp91phox (FIG. 1) is released into the bloodstream.

Moreover, high levels of soluble gp91^{phox} (hereinafter called “sgp91^{phox}”) could be detected in serum samples from patients suffering from a dysmetabolic disease, such as hypercholesterolemia (FIGS. 1 and 2A-C).

As herein used, by “soluble gp91^{phox}”, or “sgp91^{phox}”, or “serum gp91^{phox}”, or “serum sgp91^{phox}”, is meant the gp91^{phox} peptide that is released in the bloodstream upon activation of platelet NADPH oxidase and that can be detected with extreme specificity by use of the polyclonal or/and monoclonal antibody of the invention, as will be explained below.

Further to this, activation and inhibition of platelet NADPH oxidase, measured as levels of sgp91^{phox} in serum, in said patients showed a good correlation with the urinary isoprostane levels, the latter being currently considered as strictly correlated with the levels of organic oxidative stress, as described below in the Examples section.

Detection of gp91^{phox} levels present on platelet membranes, on the contrary, did not prove to be an indicator of actual NADPH oxidase activation nor was strongly correlated to urinary isoprostanes levels and, therefore, to oxidative stress in the body (FIG. 5).

The results obtained by the Inventors (shown in Examples 1 and in FIGS. 2D e 3D), and the good correlation between serum “sgp91^{phox}” levels and urinary isoprostane levels (Rs=0.77, p<0.001) calculated by univariate analysis, for the first time demonstrate that the enzymatic activity of NADPH oxidase can directly influence urinary isoprostane levels in humans.

On the other hand, the low correlation seen between the presence of gp91^{phox} on platelet membrane (measured by cytofluorimetry) and urinary isoprostanes (Rs=0.5, p=0.05), just confirms that isoprostane levels are dependent on sgp91^{phox} activity and not on the quantity of gp91^{phox} present on platelets.

Since it is known that large quantities of NADPH oxidase are found in blood phagocytic cells (such as neutrophils), the Inventors sought to define the levels of gp91^{phox} released in vitro by monocytes, neutrophiles and platelets after proper stimulation (FIG. 5). When NADPH oxidase in platelets, polymorphonuclear cells and lymphocytes/monocytes was stimulated by arachidonic acid (5C-D), phorbol myristate acetate or lipopolysaccharide respectively (5D), activation of this enzyme resulted in release of cellular gp91^{phox} into the cell culture medium, which was thus measured as sgp91^{phox} (soluble gp91^{phox}). Also, western blot analysis of platelet cell membranes (FIG. 5B) showed that upon stimulation of NADPH oxidase, the levels of membrane gp91^{phox} were reduced while the concentration of sgp91^{phox} released in the cell supernatant was considerably raised. In parallel, ROS production specifically driven in platelets by NADPH oxidase activation with arachidonic acid was confirmed by using two different specific inhibitors of the oxidase, i.e. the peptide 91phox ds-tat (Rey F E et al., Circ Res (2001) 89: 408-414 and Griendling K K et al., J Cardiovasc Pharmacol (2007) 50:9-16 and apocynin (Griendling K K et al., (2007) supra) (3A). In FIG. 3C are shown the corresponding levels of sgp91^{phox} in the supernatant of platelets: as it can clearly be seen, specific inhibition of NADPH oxidase also causes a decrease in the levels of sgp91^{phox} shed in the cell medium.

Thus, the method of the invention allows to detect the activation levels of the NADPH oxidase enzyme in the human or animal body, preferably in a mammal, more preferably in humans, by means of a simple and rapid ELISA assay which makes use of poly- or monoclonal proprietary antibodies to the catalytic subunit if said enzyme, due to the unexpected finding that platelet NADPH oxidase releases sgp91^{phox} in the bloodstream as a consequence of its activation.

In addition, the good correlation shown between the presence of soluble gp91^{phox} and the urinary isoprostanes levels allows the method of the invention to be used as a choice assay for the analysis of oxidative stress in an animal, preferably in a mammal, more preferably in humans.

By the term “plasma” it is meant a biological sample obtained by centrifugation, typically at 3000 rpm, of a certain quantity of sodium citrate-anticoagulated blood. By the term “serum” it is meant the supernatant of a sample of coagulated blood, typically kept in test tube at 37° C. in a water bath for 30 minutes and then centrifuged at 3000 rpm. By the term “supernatant” it is meant the liquid medium in which cells are grown, after the latter have been removed by e.g., centrifugation of filtration. These definition and methods are anyway known to the expert in the field.

The method of the invention relates in particular to the detection of “sgp91^{phox}” in serum and in other cell-free biological samples, such as plasma and supernatants from cell cultures free from proteins of molecular weight above 200 kDa, for example by means of traditional filtration methods or gel filtration.

In a preferred embodiment, the present invention relates to the detection of gp91^{phox} protein by an ELISA method, wherein a proprietary monoclonal antibody against the highly immunogenic peptide AERIVRGQTAESLAVHNITVCEQKISEWGKIKECPIPQFAGNPPM (corresponding to the gp91^{phox} sequence 224-268; SEQ ID NO 3) from hybridoma SD-6311 is used. This peptide was chosen because of its particular immunogenicity and because it is in an extracellular gp91^{phox} protein domain.

Said antibody was obtained by injecting mice with the purified synthetic peptide of SEQ ID NO 3, the spleen of the most responsive animal was then used for obtaining hybridomas which were screened and selected according the usual methods known to the expert in the field. The selected hybridoma was deposited at ATCC on Jun. 10, 2010 under No. SD-6311.

Alternatively, the proprietary rabbit polyclonal antibody against the peptide LNFARKRIKNPEGGLC (SEQ ID NO 1) can be used. This sequence corresponds to aminoacids 152-168 of the human gp91^{phox} which are in a portion of the protein usually exposed to the outer side of the cell membrane. This antibody was obtained by injecting rabbits with the peptide of SEQ ID NO 1 conjugated with keyhole limpet hemocyanin (KLH), according to methods known to the expert in the field.

The method of the invention can find a useful application in the field of oxidative stress control in pathologies of dysmetabolic type, such as diabetes, hypercholesterolemia and hyperlipidemia, as well as in cardiovascular pathologies, such as hypertension, atherosclerosis, cardiac hypertrophy and myocardial infarction.

The present method can also be useful in the field of sepsis and with reference to conditions characterized by an important inflammatory component, such as rheumatoid arthritis.

The method of the invention is also useful to indirectly monitor the efficacy of a therapy for one of the diseases mentioned above (Cave A. C. et al., Antiox. Redox Signal. (2006) 8:691-728; Valko M. et al., Int. J. Biochem. Cell. Biol. (2007) 39:44-84). This can be done by measuring the decrease in the oxidative stress caused by the pathology induced by the beneficial effects of the therapy.

The method of the present invention comprises the steps of:

(i) adding an antibody against gp91^{phox}, preferably directed against one peptide derived from its sequence, to a biological platelet-free fluid obtained from whole blood. Preferably the peptide is a synthetic peptide, for example the peptide having the sequence of SEQ ID NO 1 or the peptide having the sequence of SEQ ID NO 3 or a corresponding larger peptide thereof.

(ii) detecting the formation of the antibody/gp91^{phox} complex or of the antibody/gp91^{phox} peptide complex.

Preferably steps (i) to (ii) are followed by further steps of

(iii) building a calibration curve by using a standard preparation of gp91^{phox} or of corresponding peptides, preferably a preparation of the same peptide used for eliciting the antibody, and

(iv) calculating the concentration of sgp91^{phox} or of its peptides present in the assayed sample by using the result obtained in step (ii) and the calibration curve obtained in step (iv).

In a preferred embodiment, the method of the invention provides for a specific detection of the presence and quantity of protein sgp91^{phox} in serum by using immunoprecipitation followed by SDS-PAGE and Western blotting.

Said preferred embodiment of the invention comprises the steps of:

(i) Incubating a sample of biologic fluid, e.g. serum, with a specific monoclonal or polyclonal antibody against gp91^{phox};

(ii) Recovering the antibody/gp91^{phox} formed during step (ii);

(iii) Analyzing the thus obtained complex by means of SDS-PAGE and Western blotting.

In a further embodiment, the method of the invention provides for the use of the ELISA technique and comprises the steps of:
Putting said serum sample in contact with a capture monoclonal or policlonal antibody (primary antibody) against gp91^{phox} or its corresponding peptides, preferably against the peptide of SEQ ID NO 1 or of the peptide of SEQ ID NO 3 or longer peptide sequences encompassing said sequences;
Incubating with a secondary peroxidase conjugated antibody;
Revealing by incubating with an adequate substrate;
Reading the color obtained with a spectrometer; and
Calculating the result by means of a calibration curve obtained by using increasing concentrations of gp91phox or of the peptide(s) used for eliciting the primary antibody.

The advantages of the method resides in its ease of use, as it is carried out in platelet-free fluids, and it can be executed as a simple ELISA method, with no need for a particular training of the operating staff and with no need for expensive working equipment.

In addition, due to the observation made by the Inventors that activated NADPH oxidase in circulating platelets causes the release of sgp91^{phox}, and due to the observed and proven correlation between this release and the urinary isoprostanes levels, the method of the present invention is the very first one, and, currently, the only analytical method enabling a direct measure of oxidative stress levels.

The method of the invention is therefore particularly useful for studying variations in the oxidative stress in some dysmetabolic diseases (such as diabetes, hyperglycemia, obesity, hypercholesterolemia and hypertrigliceridemia), in sepsis, in inflammatory diseases (for example rheumatoid arthritis), in chronic infections, and in cardiovascular pathologies, such as hypertension, atherosclerosis, cardiac hypertrophy and myocardial infarction).

The following examples are to be considered as illustrating the invention and should not be construed as a limitation of its scope.

### EXAMPLES

### Example 1

### Clinical Study in Hypercholesterolemic Patients

This clinical study was authorized by the Ethical Committee of the University of Rome “La Sapienza”.

### Patients and Exclusion Criteria

The study was carrier out in 30 consecutive patients (16 male and 14 female subjects) suffering from hypercholesterolemia, defined as LDL cholesterol levels above 200 mg/dl, 52±4 years of age.

The exclusion criteria were renal insufficiency, serious kidney conditions (serum creatinine levels>2.5 mg/dl), diabetes mellitus, artherial hypertension, a stry of or presence of myocardial infarction or other atherothrombotic conditions, any autoimmune disease, cancer, recent or ongoing infections. Moreover, patients taking non-steroidal antinflammatory drugs (NSAIDs), cholesterol metabolism interfering drugs or vitamine supplements were also excluded from the study.

All the patients were all Caucasian coming from the same geographical area.

### Study Design

The data coming from all the subjects included in the study were used to carry out a comparative analysis between urinary isoprostanes levels and the levels of gp91^{phox} detected by means of immunoprecipitation from serum samples

Hypercholesterolemic patients were openly randomized to be treated with a 30-day therapy with atorvastatin (10 mg/day) together with a proper diet, or to treatment with proper diet only.

During this phase of the study the patients were subjected to a low-fat diet containing average quantities of macronutrients that corresponded to a 7% energy coming from fats and 200 mg/day of cholesterol coming from diet, according to ATPIII guidelines.

The randomization numbers were randomly given by a medical doctor who was not participating in the study, who also kept the key in a sealed envelope during the whole duration of the study. Randomization was done according to a procedure based, on a casual number sequence. The medical doctors who were involved in the study did not know the treatment allocation.

The principal investigator proceeded with the opening of the randomization list only at the end of the study and after the laboratory tests were completed.

### Laboratory Tests

Whole blood anticoagulated samples were from fasting patients (5 ml) in between 8.00 and 9.00 in the morning. The samples were then kept for 1 hour at 37° C. and centrifuged at 3000×g for 10 minutes to obtain sera. The supernatant thus obtained was kept frozen at −80° C. until used for the assays.

10 ml aliquots of the morning urine samples were kept frozen at −80° C. until used for the assays

The following assays were performed using 1 ml of serum and 10 ml of urine:
Total cholesterol (TC), triglicerides (TG) and HDL cholesterol after precipitation with phosphotungstic acid/MgCl₂ with enzymatic commercial methods (DADE Behringer);
LDL cholesterol according to Friedewald formula;
urinary 8-iso prostaglandin F2α (PGF2α-III) by the validated EIA method described in Hoffman S W et al., J. Neurosci. Methods (1996) 68:133-136 and in Wang Z. et al., Pharmacol. Exp. Ther. (1995) 275:94-100.

Briefly, a 10 ml aliquot of urine was extracted on a C-18 SPE column and the recovery was verified by addition of a radioactive tracer (tritiated PGF2α-III). Eluates were dried under nitrogen, resuspended in 1 ml of EIA eluting buffer (Cayman Chemical) and assayed with a specific EIA kit for PGF2α-III (Cayman Chemical). The concentration of PGF2α-III was corrected for the recovery and creatinine excretion, and was expressed as picogram per milligram (pg/ml) of creatinine.

### gp91^{phox} Detection in Human Serum

For the SDS-PAGE and western blot analysis, samples to be tested were prepared according to the following steps:

1) Immunoprecipitating the serum sample;

2) Removing the undesired antibodies from the sample:

3) Incubating the sample of step 2) with the specific monoclonal or polyclonal antibody against gp91^{phox};

4) Recovering the antibody/gp91^{phox} formed in step 3);

5) Analyzing the sample obtained in step 4) by means of SDS-PAGE and western blot.

Human serum gp91^{phox} was immunoprecipitated as follows. Serum samples were incubated overnight at 4° C. with the monoclonal antibody against gp91^{phox} from Santa Cruz Biotechnology, Inc. (catalogue sc-74514) which recognizes the protein peptide QTAESLAVHNITVCEQKISEWGKIKECPIPQFAGNPPMTWKWIVGP MFLYLCERLVRFWR (SEQ ID NO 2), corresponding to aa sequence 231-290 of the human peptide.

Step 2) of the method was carried out as follows. Agarose-conjugated protein G (code 17-6002-35 GE Healthcare) was washed 3× with PBS (phosphate buffered saline) 1× by adding 1 ml of buffer to 100 μl and centrifuging at 12.000 g for 20 minutes. The supernatant was discarded and the pellet was incubated with 500 μl of a solution PBS/BSA 5% (BSA=bovine serum albumin). 200 ml of immunoprecipitated serum prepared as in step 1) were incubated with 800 μl of RIPA buffer (Sigma Chemicals) and 50 μl of agarose-conjugated protein G for 1 h at 4° C. under constant agitation. The sample was then centrifuged at 12.000 g for 20 minutes. The supernatant thus prepared was free from unwanted antibodies and was used for isolating the immune complex (step 3)). To 500 μl of this supernatant were added 25 μl of the specific monoclonal antibody against gp91^{phox} (Santa Cruz Biotechnology) described above, and were incubated for 1 h at 4° C. under constant agitation. Control samples were prepared as follows.

a) a first control sample was prepared by adding 10 μl of isotypic antibody to 500 μl of sample;

b) a second control sample contained the supernatant only;

c) a third control sample was prepared by adding 10 μl of monoclonal antibody against gp91^{phox} to 500 μl of RIPA buffer.

Precipitation of the immune complex was carried out by adding 50 μl of agarose-conjugated protein G to all the samples and by incubating for 1 h at 4° C. The samples were then centrifuged for 20 minutes at 12.000 g. The pellet was washed 3× with RIPA buffer and once with PBS and centrifuged each time for 20 minutes at 12.000 g and discarding the supernatant. The immune complex thus formed were then isolated by affinity chromatography on ImmunoPure protein A-conjugated resin (Santa Cruz Biotechnology, Inc.).

The samples thus obtained were stored at −20° C. overnight and then analyzed by SDS-PAGE and immunoblot. After protein concentration was determined with the Bradford assay (FLUKA), 130 μg of protein per sample was resuspended in 30 μl of Laemmli sample buffer containing 2-mercaptoethanol and boiled for 5 minutes to make the beads dissociate from the immune complex. The thus solubilized samples were separated by electrophoresis on a 10% polyacrylamide gel according to the standard methods.

After separation the gels were blotted onto Immobilon (Biorad) membranes according the standard protocol known to the expert in the field, and the blotted membranes were revealed by Ponceau-S red (Sigma Chemicals) and subsequently destained by washing 4× (NaCl 32 g, KCl 8 g, Tris 12.1 g, 1000 ml H₂O), with washing buffer 1× (250 ml of washing buffer 4×, 750 ml H₂O, Tween 20 0.5%, Albumin 0.1%, pH 7.5). Finally, membranes were blocked with washing buffer 1×+5% albumin. After 5 washes of 5 minutes each with washing buffer, the membranes were incubated with the Santa Cruz monoclonal antibody against gp91^{phox} (2 μg/ml) overnight at 4° C.

After further 5 washes with washing buffer for 10 minutes each, membranes were incubated with a secondary polyclonal goat antibody against mouse IgG conjugated with 2 μg/μl horseradish peroxidase (goat anti-mouse IgG-HRP; Santa Cruz) for 1 h at room temperature. After incubation, the membranes were washed again with washing buffer for 5 times and then exposed to 4 ml ECL (2 ml oxygen peroxide+2 ml luminol/enhancer; Biorad) for 3 minutes in a dark room. Finally, the membranes were placed in contact with chemiluminescence film (Sigma, 13×18 cm) for 5 minutes. The films were then developed as known by the expert in the art using Sigma developing and fixing solutions.

The bands obtained were evaluated by means of densitometric analysis in an NIHimage 1.62 analyzer and the values expressed as Arbitrary Units (A.U.).

Platelets Preparation from Whole Blood for the Evaluation of gp91^{phox} Presence.

To obtain platelet rich plasma (PRP) from HC and HS patients, samples were centrifuged 15 min at 180 g. To avoid leukocyte contamination, only the top 75% of the PRP was collected. Platelet pellet was suspended in HEPES buffer, pH 7.4 (2×10⁸ platelets/mL, unless otherwise noted) and processed differently according to the type of experiment to be carried out.

Human Polymorphonuclear Leukocytes Preparation from Whole Blood for the Evaluation of gp91^{phox} Presence

Polymorphonuclear leukocytes (PMN) were isolated from freshly taken EDTA-blood from healthy volunteers (n=5, healthy subjects) by dextran enhanced sedimentation of red blood cells, Ficoll-Histopaque density centrifugation, lysis of remaining erythrocytes with distilled water and washing of cells with Hank's balanced salt solution (HBSS) in the absence of any divalent cations. Finally, the cell pellet was suspended in 1 ml of HBSS and stimulated with or without 10 μM of phorbol 12-myristate 13-acetate (PMA). To evaluate sgp91phox in PMN the supernatant was analyzed by ELISA method as above reported.

Lynphocytes/Monocytes Preparation from Whole Blood for the Evaluation of the Presence of gp91^{phox}

Blood samples were collected in heparinized tubes (10 IU/ml). Lynphocytes/Monocyte were isolated after centrifugation of the blood from healthy volunteers (n=5, healthy subjects) with a polysucrose-sodium diatrizoate solution, 1.077 g/ml density and 280 mOsm osmolarity (Lymphoprep; Nycomed, Oslo, Norway) at 800 g at 20° C. The Lynphocytes/Monocyte cell layer was collected and the cells were thus washed two times in a solution of cold phosphate-buffered saline (pH 7.2), supplemented with 1% fetal calf serum and 2 mmol/l EDTA (Sigma-Aldrich, Milano, Italy). The cell suspension was stimulated with or without lipopolysaccharide (100 ng/ml) (LPS), sgp91 content in the supernatant was evaluated by ELISA method as above reported.

### Evaluation of the gp91^{phox} Expression on Platelets

To evaluate gp91^{phox} expression on platelets, 50 μl of stabilized whole blood (see above) were incubated for 30 minutes with 5 μl of monoclonal antibody against gp91^{phox} (20 μg/ml; Santa Cruz), and 5 μl of PE-conjugated monoclonal antibody against CD61 (Coulter) (20 μg/ml), respectively, as described in Martino F. et al., PEDIATRICS (2008), supra. The samples incubated with the monoclonal antibody against gp91^{phox} were then incubated with a secondary IgG FITC-conjugated antibody against mouse. After incubation, 1 ml of 1× PBS was added to proceed with the cytofluorimetric analysis.

Isotypic controls were carried out by preparing a sample with a non-specific anti-mouse IgG-FITC e IgG1-PE with the same ratio f/p for FITC e PE. The adequate concentration of the monoclonal antibodies used was determined in preliminary tests (not shown).

### Cytofluorimetric Analysis

All the samples were analyzed within 15 minutes from dilution. FITC fluorescence was detected with an Epics XL-MCL cytometer (Coulter, Milan Italy) at 525 nm, while PE fluorescence was detected at 575 nm. All the parameters were grouped with a 4-decade logarithmic amplification.

Spectral overlap was balanced by fluorescence compensation, as defined in preliminary tests. Cytometer settings were checked with Flow-Chek Fluorospheres. Platelets and platelet-platelet aggregates were identified by logical gating according to their CD61 phycoerythrin fluorescence and forward scatter characteristics. A threshold of 0.5% FITC-positive events was set in the first isotype control of each subject. Analysis was stopped automatically after the measurement of 50 000 events. Intra-assay and interassay coefficients of variation were 1.0% and 0.2%, respectively.

### Statistical Analysis

Categorical variables were shown as percentage and the continuous variables as average+SD (Standard Deviation).

The independence of the categorical variables was tested by means of the χ² test. The comparisons between HC patients and healthy subjects were carried out by means of the Student T test and were replicated, as appropriate, with non-parametric tests ((z) test of Kolmogorov-Smirnov) in case of non-homogeneous variances, as verified by the Levene test.

Bonferroni's correction was applied to take into account the increase in type-I error due to the multiple assays.

The correlation analysis was carried out by means of Pearson's test. A value of P>0.05 was considered as statistically significant. The data from the clinical study were analyzed to verify the effects of treatment on gp91^{phox}, total cholesterol and on urinary isoprostanes, applying MANOVA analysis with a factor among subjects (treatment group) and an internal factor (two time points: 0 and 30 days from treatment start).

The covariates considered were the possible casual differences in age, sex and blood systolic and diastolic pressure between the two groups (the treatment by diet arm and the treatment by diet+atorvastatin arm).

The statistical analysis was carried out with SPSS 13.0 software for Windows.

Calculation of sample size—As mentioned above, in the clinical study were enrolled all those patients who complied with all the inclusion/exclusion criteria. The number of control patients (n=20) was calculated by means of a two-tailed Student T test for independent groups, taking into account:
the clinically relevant difference in the gp91^{phox} to be measured (d) as ≧100 A.U. (Arbitrary Units);
homogeneous standard deviations between groups, SD=50 A.U.
the probability of type-I error a=0.05 and potency 1−b=0.90.

These assumptions led to the calculation of the sample size as n=19/group.

As to the therapeutical intervention group, the minimum sample size was calculated by means of a two-tailed Student T test for a sample, taking into account:
the clinically relevant difference in the gp91phox levels to be measured (d) as 50 A.U.;
homogeneous standard deviations between groups, SD=50 A.U.;
the probability of type-I error a1/4=0.05 and the potency 1−b=0.90.

These assumptions led to the calculation of the sample size as n=8/group.

### Results

In FIG. 1 are shown the results obtained by separating with SDS-PAGE the immunoprecipitates from three healthy subjects sera. The 105 kDa band was present as a background in all the sera samples analyzed. On the contrary, the 91kDa band was specifically recognized by the Santa Cruz monoclonal antibody against the peptide of SEQ ID NO 2 of gp91^{phox}.

In Table 1 the demographic and clinical features of the subjects participating in the clinical study and the laboratory results obtained are shown. The two groups of patients involved, i.e. hypercholesterolemic patients (HC) and healthy subjects (HS), did not show relevant differences in terms of age, sex, body mass index (BMI), smoking habit, fasting glucose levels and blood diastolic and systolic pressure. Hypercholesterolemic patients showed, as expected, significantly higher serum total cholesterol (TC) LDL cholesterol (LDL-C and triglyceride (TG) levels (p<0.001). HDL cholesterol levels (HDL-C) were significantly higher in hypercholesterolemic patients than in healthy subjects.

TABLE 1
Clinical parameters of hypercholesterolemic patients and of
healthy subject enrolled in the clinical study

| | Hyper- | Healthy | |
|---|---|---|---|
| | cholesterolemic | subjects | |
| Parameters | subjects (n = 30) | (n = 20) | P value |
| Age (years)* | 52.5 ± 3.8 | 52 ± 3 | 0.277 |
| Gender (male/female) | 16/14 | 10/10 | 0.954 |
| BMI** (kg/m²)* | 25.4 ± 2.5 | 25.7 ± 2.4 | 0.628 |
| Systolic Blood Pressure | 127 ± 12 | 125 ± 11 | 0.924 |
| (mmHg)* | | | |
| Diastolic Blood Pressure | 75 ± 9 | 75 ± 10 | 0.928 |
| (mmHg)* | | | |
| Smokers | 3 | 2 | 0.630 |
| Total Cholesterol (mg/dL)* | 278 ± 39 | 187 ± 11 | 0.001 |
| LDL cholesterol (mg/dL)* | 187 ± 13 | 98 ± 14 | 0.001 |
| HDL cholesterol (mg/dL)* | 62 ± 11 | 50 ± 11 | 0.001 |
| Triglicerides (mg/dL) | 103 ± 21 | 73 ± 15 | 0.001 |
| Fasting blood glucose levels | 84 ± 12 | 84 ± 12 | 0.961 |
| (mg/dL)* | | | |
| Urinary Isoprostanes (pg/mg | 366 ± 63 | 130 ± 38 | 0.001 |
| creatinine)* | | | |
| gp91^{phox} (A.U.) | 199 ± 58 | 25 ± 30 | 0.001 |
| gp91^{phox} (M.F.) | 6.9 ± 1.6 | 3.4 ± 1.1 | 0.001 |

*Data are expressed as average ± SD
**BMI = Body Mass Index
M.F = MEAN FLUORESCENCE.

HC patients showed an increased oxidative stress as judged on the basis of the urinary isoprostanes levels (Table 1, FIG. 2D), from the increased serum gp91^{phox} (sgp91^{phox}) with respect to the control samples (Table 1; FIGS. 2A and B) and from the platelet gp91^{phox} levels (Table 1; FIG. 2C). According to the bivariate analysis, the sgp91^{phox} showed a significant correlation with serum cholesterol levels (Rs=0.52, p<0.001) and with urinary isoprostane levels (Rs=0.57, p=0.05).

The excretion of isoprostanes also showed a significantly correlation with serum cholesterol levels (Rs=0.59, p<0.001).

Regarding the clinical intervention study, the patients randomized to the treatment by diet (group A) and those randomized to treatment by diet+atorvastatin (10 mg/die; group B) showed similar total cholesterol levels, sgp91^{phox} and urinary isoprostane levels at time 0 (Table 2 and FIG. 3).

TABLE 2
Clinical study; basal parameters of HC patients randomized to
treatment by diet (group A) or to diet + atorvastatin (group B)

| | Group A | Group B | |
|---|---|---|---|
| Parameters | (n = 15) | (n = 15) | P value |
| Age (years)* | 52.8 ± 3.7 | 52.2 ± 4.1 | 0.677 |
| Gender (male/female) | 8/7 | 8/7 | 0.714 |
| BMI** (kg/m²)* | 25.1 ± 2.4 | 25.7 ± 2.6 | 0.502 |
| Systolic Blood Pressure (mmHg)* | 128 ± 12 | 126 ± 12 | 0.661 |
| Diastolic Blood Pressure (mmHg)* | 75 ± 9 | 75 ± 10 | 0.660 |
| Smokers | 1 | 2 | 1.000 |
| Fasting blood glucose levels (mg/dL)* | 84 ± 12 | 84 ± 12 | 0.720 |
| Triglicerides (mg/dL) | 102 ± 19 | 103 ± 24 | 0.965 |
| Total Cholesterol (mg/dL)* | 280 ± 32 | 276 ± 46 | 0.796 |
| Urinary Isoprostanes (pg/mg | 348 ± 69 | 383 ± 51 | 0.129 |
| creatinine)* | | | |
| Gp91^{phox} (A.U.) | 187 ± 46 | 211 ± 68 | 0.261 |

*Data are expressed as average ± SD.

At the end of 30 days of treatment, group B showed a significant reduction of sgp91^{phox} (from 211±68 to 154±43 A.U., p=0.035) (data not shown) together with a significant reduction in the urinary isoprostanes levels (from 383±51 to 241±58 pg/mg of creatinine, p<0.001) (FIG. 3D) and of serum total cholesterol (from 276±46 to 208±38 mg/dl, p<0.001) (data not shown). On the contrary, group A showed only a weak reduction in total cholesterol levels (from 280±31 to 261±15 mg/di, p=0.045).

MANOVA analysis of the data confirmed the significance of the interaction between the treatment time and group variables, pointing to a significant effect of different treatments on the presence of sgp91^{phox} [F(1,21)=5.6, p=0.02], of the urinary isoprostanes [F(1,21)=66.1, p=0.01] and of total cholesterol [F(1,21)=9.6, p=0.01]. No significant correlation was found between treatment time and the other covariates, such as age, smoking habits, sex, blood pressure; etc.

### Example 2

### Evaluation of sgp91^{phox} with the ELISA Assay of the Invention in the Clinical Study with Atorvastatin

A correlation was found between sgp91^{phox} and the urinary isoprostanes by the sandwich ELISA assay of the invention in the control and HC subjects in the clinical study.

The samples tested were sera from the same sampling of the clinical study of Example 2, stored frozen at −80° C. up to the use.

The peptides LNFARKRIKNPEGGLC (SEQ ID NO 1) or AERIVRGQTAESLAVHNITVCEQKISEWGKIKECPIPQFAGNPPM (SEQ ID NO 3) of gp91^{phox} respectively, were used as the standard.

The proprietary primary policlonal antibody against SEQ ID NO 1 and the primary monoclonal antibody against SEQ ID NO 3 (i.e., the proprietary antibody of the present invention from the hybridoma deposited at ATCC on Jun. 10, 2010 under No. SD-6311) already described above were diluted 1:100 with coating buffer (catalog. C3041, Sigma Chemicals) and 100 μl of this primary antibody solution were placed in 96-well ELISA plates for 1 h at room temperature (RT). After incubation the content of each well in the plates was removed and the wells were washed three times with washing buffer (Tris-buffered saline 50 mM, pH 8.0, Tween 20; catalog. T9039, Sigma Chemicals).

Subsequently, 200 μl of saturation buffer containing 1% BSA (catalog T6789, Sigma Chemicals) were added to the wells for 30 minutes at RT. The wells were then washed three times with the washing buffer, as above.

100 μl of standard or of sample to be assayed were then added to the wells and the incubation was carried out for 1 h at RT.

A standard curve was obtained by using increasing concentrations of the gp91^{phox} peptide of SEQ ID NO 1 or of the peptide of SEQ ID NO 3 (i.e., 64 pg/ml; 32 pg/ml; 16 pg/ml; 8 pg/ml) obtained by diluting the peptides in buffer, and incubated for 1 h see FIG. 4, panel A and panel B.

The wells were then aspirated and were then washed three times with the washing buffer, as described above.

100 μl of a secondary goat-anti-rabbit IgG-HRP antibody (Santa Cruz, catalog sc2004) or of a secondary goat anti-mouse IgG-HRP antibody (Santa Cruz, catalog sc 2060) diluted 1:100 with coating buffer, were then incubated at RT for 1 h.

After aspirating the contents of the wells and washing three times with washing buffer, as described above, 100 μl of substrate (Santa Cruz, catalog SK-4400) were added to the wells for 30 minutes at RT. At the end of the incubation, 100 μl of H₂SO₄ 2M (Merck, catalog 30148297) were also added to the wells. Subsequently, reading of the developed color was carried out with a spectrophotometer at 450 nm.

The final concentration of the samples were calculated by using the proper standard curve obtained with a gp91^{phox} peptide, as described above.

In Table 3 and 4 the steps described above for performing the ELISA assay with the polyclonal antibody and the monoclonal antibody of the invention, respectively are schematically reported.

TABLE 3

| Step | Sample/Antibody | Assay Conditions |
|---|---|---|
| Coating with | Anti-gp91^{phox} pAb* | 100 μl 4 μg/ml/well, |
| primary Ab | | 1 h, RT |
| Sample | 100 μl Serum | 1 h, RT |
| Secondary Ab | 100 μl goat | 100 μl dilution Ab 1:2000, |
| | anti-rabbit IgG1-HRP | 1 h, RT |
| | (Santa Cruz sc-2004) | |
| Revelation | TMB (Santa Cruz sk-4400) | 405 nm/450 nm, 15 min |

The zero threshold was established at 0.015 pg/ml
pAb* = polyclonal antibody;
RT = room temperature

TABLE 4

| Step | Sample/Antibody | Assay Conditions |
|---|---|---|
| Coating with | Anti-gp91^{phox} mAb | 100 μl 4 μg/ml/well, |
| primary Ab | | 1 h, RT |
| Sample | Siero 100 μl | 1 h, RT |
| Secondary Ab | 100 μl goa | 100 μl dilution Ab 1:2000, |
| | t anti-mouse IgG-HRP | 1 h, RT |
| | (Santa Cruz sc-2060) | |
| Revelation | TMB (Santa Cruz sk-4400) | 405 nm/450 nm, 15 min |

The zero threshold was established at 0.015 pg/ml
mAb* = monoclonal antibody;
RT = room temperature

By using the polyclonal antibody of the invention in the ELISA assay described above to detect sgp91^{phox} in the same samples from the patients of the clinical study described in Example 1, it was possible to see an increase of this protein concentration in the sera from HC patients with respect to the healthy subjects (FIG. 3A). Similar results were obtained using the monoclonal antibody of the invention (FIG. 3B). As shown in FIGS. 3A and B, group B showed a significant reduction in the sgp91^{phox} both measured by the polyclonal and the monoclonal antibody (−33%, from 36.6±5.6 to 24.5±7.7 pg/ml, p<0.001) and (−25%, from 32.5±4.6 to 23.5±6.5 pg/ml, p<0.001) at the end of the treatment with atorvastatin (30 days). On the contrary, no significant variation in platelet gp91^{phox} was seen in both groups after treatment (FIG. 3C).

A significant correlation between the sgp91^{phox} levels detected by western blot (by using the Santa Cruz commercial monoclonal antibody as described above) and the levels detected by ELISA was also observed, both by using the polyclonal antibody (Rs=0.61, p<0.001) and by using the monoclonal antibody of the invention (RS=0.70, p<0.001) (data not shown).

The sgp91^{phox} concentration as detected by use of the polyclonal and monoclonal antibody of the invention, showed a significant correlation with the urinary isoprostanes levels (Rs=0.61, p<0.001) and (Rs=0.71, p<0.001) and with the blood cholesterol concentration (Rs=0.52, p<0.001) and (Rs=0.61, p<0.001).

In this regard, it is interesting to point out that the levels of sgp91^{phox} as measured by using the Santa Cruz monoclonal against SEQ ID NO.2 showed a less significant correlation with urinary isoprostane levels (Rs=0.57, p=0.05) with respect to the correlation seen with the monoclonal antibody of the invention against SEQ ID NO.3 of gp91^{phox} (Rs=0.71, p<0.001). This in agreement with the hypothesis that, upon activation of platelet NADPH oxidase on the cell membrane, gp91^{phox} is cleaved and thus the extracellular moiety of the protein is released into the bloodstream.

The currently used method of detection of gp91^{phox} in platelets by immunocytofluorimetry was shown by the Inventors to be much less significant than the detection of sgp91^{phox} with respect to the correlation with urinary isoprostanes (Rs=0.5, p=0.05 vs Rs=0.71, p<0.001). This means that detection of sgp91^{phox} by ELISA using the antibodies of the invention, both the polyclonal and the monoclonal one, against different peptides of gp91^{phox} protein, seems to be the analytic method most reflective of the gp91^{phox} activation and of the variations in oxidative stress among the known assays.

In the clinical study, the patients randomized to group A (diet) and those randomized to group B (diet+atorvastatin 10 ng/die) showed at baseline sgp91^{phox} levels very similar between them, as measured by ELISA using both the polyclonal and the monoclonal antibodies of the invention (FIG. 3A-B).

### Example 3

### Evaluation of NADPH Oxidase Activation

To test NADPH oxidase activity, the levels of ROS in platelets from 5 human healthy subjects were measured by cytofluorimetry in the presence and in the absence of specific NADPH oxidase inhibitors, such as apocynin and the gp91^{phox} blocking peptide gp91ds-tat. Platelets were incubated with 2′,7′-dichlorofluorescin diacetate 5 mM for 15 minutes at 37° C. After incubation, 100 μl of the sample was treated with arachidonic acid (AA, 0.5 mM) in presence or less of apocynin (100 μM) or the gp91^{phox} specific blocking peptide (gp91ds-tat, 50 μM). Then 10 μl of each sample was diluted with 1 ml of PBS and analyzed by flow cytometry. Basal OFR level in resting platelets was expressed as mean fluorescence (MF), AA-induced OFR production was expressed as stimulation index (S.I.=mean level of fluorescence in AA-stimulated platelet/mean level of fluorescence in unstimulated platelets) (Pignatelli P et al. Blood (1998) 95:484-490.

As shown in FIG. 5A, the results obtained showed an increase in ROS production after platelet activation with arachidonic acid (AA) which was inhibited by apocynin and gp91ds-tat. In FIG. 5B is shown the effect of the stimulation with AA and of the inhibition with apocynin and gp91ds-tat on the gp91phox present on the platelet membrane evaluated with monoclonal antibody of the invention. Finally, the levels of sgp91^{phox} in the supernatant of the same platelets treated with apocynin and gp91ds-tat, as detected by ELISA assay using the monoclonal antibody of the invention clearly showed that modulation of the platelet NADPH activity and of its gp91^{phox} subunit by specific inhibitors directly influences the release of sgp91^{phox} in the medium (FIG. 5C).

This results reflects the fact that activation of NADPH oxidase induced ROS formation and subsequent related release of sgp91^{phox} in the extracellular medium.

sgp91^{phox} is Released by Different Blood Cells in Addition to Platelets

To evaluate the source of sgp91phox we isolated platelets, PMN and lymphocytes/monocytes from the same blood sample. Cell suspension in PBS was stimulated with AA for platelets, PMA for PMN and LPS for lymphocytes/monocytes as reported above; the supernatant sgp91^{phox} content was detected by ELISA assay with the monoclonal antibody of the invention. sgp91phox were 1.18±0.84 pg/ml in unstimulated and 7.05±2.04 pg/ml in AA-stimulated platelets.

In PMA-stimulated PMN, sgp91phox were 11.85±3.23 pg/ml vs 1.53±0.66 pg/ml in unstimulated PMN.

In LPS-stimulated lymphocytes/monocytes sgp91phox were 7.5±2.64 pg/ml vs. 1.11±0.55 pg/ml in unstimulated samples (FIG. 5D). The sum of sgp91phox released from activated platelets, PMN and monocytes was 31.8 pg/ml, which corresponded to >90% of the sgp91^{phox} of the whole serum sample (35.42±2.87 pg/ml) (FIG. 5D).

1. A method for in vitro detecting the activation of NADPH oxidase enzyme by measuring the levels of soluble gp91P^{hox} as a marker of oxidative stress, to be carried out in a biological fluid comprising a serum, a plasma, a cell culture supernatant or a cellular lysate, said method comprising the steps of:
(i) providing a biological fluid, and Putting the biological fluid in contact with an antibody that specifically binds to gp91P^{hox} or corresponding peptides thereof, and optionally the a peptide has a sequence as set forth in SEQ ID NO 1 or as set forth in SEQ ID NO 3 or corresponding mixtures thereof;
(ii) Detecting the formation of the complex antibody/gp91P^{hox} or antibody/gp91P^{hox} peptide.

2. A method for evaluating the efficacy of a therapy with anti-inflammatory agents or antihyperglicemic agents in patients suffering from arthritis or diabetes, or a therapy for a cardiovascular disease, or a therapy with statins in hypercholesterolemic patients, said method comprising the detection of a variation in the activation of NADPH oxidase by the method according to claim 1,
wherein an increase in the amount (levels of) soluble gp91P^{hox} indicates activation of NADPH oxidase and indicates increased oxidative stress,
wherein optionally decreased oxidative stress indicates increased efficacy of the therapy.

3. An isolated or recombinant Extracellular moiety of gp91P^{hox} for use in detecting the activation of NADPH oxidase.

4. An isolated or recombinant peptide having a sequence as set forth in SEQ ID NO 1, or SEQ ID NO 3, or a peptide sequence consisting of SEQ ID NO:1 or SEQ ID NO:3, to be used for the detection of an oxidative stress.

5. An isolated or recombinant nucleotide sequence codifying for a peptide of claim 4.

6. A polyclonal or monoclonal antibody against the peptides according to claim 4.

7. The monoclonal antibody according to claim 5, obtained from the hybridoma deposited at the American Type Culture Collection (ATCC) on Jun. 10, 2010 under No. SD-6311.

8. A Hybridoma No. SD-6311, deposited at the American Type Culture Collection (ATCC) on Jun. 10, 2010.

9. A kit to carry out the method according to claim 1, comprising the monoclonal antibody from the hybridoma SD-6311.

10. The kit according to claim 9, also comprising reagents conventionally used in ELISA methods.

11-15. (canceled)

16. The method of claim 1, wherein the method further comprises the following additional steps:
(iii) Building a calibration curve using a standard preparation of gp91^{phox}, and optionally the peptide used to elicit the antibody comprises a peptide as set forth in SEQ ID NO 1 or SEQ ID NO 3; and
(iv) Calculating the concentration of gp91P^{hox} or corresponding peptides thereof in the sample using the calibration curve of step (iii).

17. The method of claim 2, wherein the therapy is a therapy for a sepsis.

18. The method of claim 2, wherein the therapy for a cardiovascular disease is a therapy for a hypertension, an atherosclerosis, a cardiac hypertrophy or a myocardial infarction.
